(19) United States
(12) Patent Application Publication
(10) Pub. No.: US 2022/0414436 A1
(43) Pub. Date: Dec. 29, 2022

(54) SYNTHESIS OF BRANCHING MORPHOLOGIES

(71) Applicant: Ecole Polytechnique Federale De Lausanne (EPFL), Lausanne (CH)

(72) Inventors: Lida Kanari, Bussigny (CH); Kathryn Pamela Bellwald, Aigle (CH); Henry Markram, Pully (CH)

(21) Appl. No.: 17/770,172

(22) PCT Filed: Oct. 13, 2020
(86) PCT No.: PCT/EP2020/078777

§ 371 Date: Apr. 19, 2022

(30) Foreign Application Priority Data
Oct. 21, 2019 (GR) 20190100470

(51) Int. Cl. G06N 3/06

(52) U.S. Cl. CPC G06N 3/061

(57) ABSTRACT

Methods, systems, and apparatus, including computer programs encoded on a computer storage medium for generating model neurons. In one aspect, a method includes receiving a plurality of descriptions of branches of dendrites of one or more neurons and generating a collection of model neurites. Each of the descriptions characterizes, for an individual branch, i) a distance from a cell body at which the individual branch first bifurcates and ii) a distance from the cell body at which the individual branch actually terminates. Generating the collection of model neurites includes repeatedly selecting a description of a branch from the plurality and probabilistically generating a topology of a model neurite based on the selected description. The probabilistic generation of the model neurite includes deciding whether to bifurcate, terminate, or continue the model neurites at different positions based on the selected description.

### BACKGROUND

This invention relates to the generation of models of neuronal dendrites and other branching morphologies.

Many real-world biological systems include branching structures that exhibit a wide variety of shapes. Examples include tree roots and the dendrites of neurons. In many contexts, it would be desirable to model or simulate such branching structures. For example, scientific modelling can be used to investigate the properties of branching structures when, e.g., it is impractical or unethical to make direct measurements on the real-world systems themselves.

Modeling such branching structures is an inherently difficult problem for several reasons. For example, if the model reflects too many characteristics of the branching structures, computational complexity can expand rapidly. On the other hand, if the model reflect too few or only irrelevant characteristics of the real-world structures, then the relevant properties of the branching structures may not be derivable from the model.

### SUMMARY

The present document describes methods and systems for generating models of branching morphologies such as neurons. The methods and systems can lever a topological description of a relatively small number of example branches to generate large numbers of biologically relevant model morphologies.

In one aspect, methods, systems, and apparatus, including computer programs encoded on a computer storage medium, for synthesizing models of branching morphologies are described. For example, such a method includes receiving a persistence barcode that characterizes a plurality of biological branches and generating a collection of model branches, including repeatedly selecting a bar from the persistence barcode and probabilistically generating a topology of a model branch based on the selected bar. The bars in the persistence barcode represent positions of bifurcations and terminations of the biological branches. Each bar in the persistence barcode characterizes a single of the biological branches. The probabilistic generation of the model branch includes deciding whether to bifurcate, terminate, or continue the model branch at different positions. The bifurcation probability is based on a position of a bifurcation in the selected bar and the termination probability is based on a position of a termination in the selected bar.

This and other aspects can include one or more of the following features. The persistence barcode can characterize the biological branches of a single tree. The bars in the barcode can encode radial distance from a source of a trunk of the tree. In response to deciding to bifurcate a first branch based on a first selected bar, the method can include selecting a second bar from a subset of the bars in the persistence barcode, wherein the subset of the bars excludes the first selected bar. At least some of the bars of the persistence barcode can include a bifurcation angle that characterizes an angle at which a component emerges from the biological branch characterized by those bars. The method can also include determining directions of daughter branches based on the bifurcation angles. The bifurcation probability and the termination probability can be sampled from an exponential distribution exp (−λx), wherein x is the position of the bifurcation or the position of the termination and λ between 10% and 1000% of a separation between successive distances at which the decisions whether to bifurcate, terminate, or continue are made. The biological branches can be neurites.

In another aspect, methods, systems, and apparatus, including computer programs encoded on a computer storage medium, for generating model neurons are described. For example, such a method includes selecting, on a cell body of the model neuron, a site from which a model neurite is to project from the cell body, and probabilistically generating the model neurite, including, for each of a successive plurality of distances from the cell body, deciding whether to i) bifurcate the neurite, ii) terminate the neurite, or iii) continue the neurite for another step, wherein a probability of bifurcation, a probability of termination, and a probability of continuation are functions of the distance from the cell body.

This and other aspects can include one or more of the following features. The probability of bifurcation can be calculated based on a distance from the cell body at which a first branch of a neuronal dendrite first bifurcates. The probability of termination can be calculated based on a distance from the cell body at which the branch of the neuronal dendrite actually terminates. The probability of bifurcation and the probability of termination are sampled from an exponential distribution exp (−λx), wherein: x is the distance from the cell body and λ is between 10% and 1000% of a separation between successive distances at which the decisions whether to bifurcate, terminate, or continue are made. Selecting the site from which the neurite is to project can include selecting the site based on a second site from which a second neurite projects from the cell body. Selecting the site from which the neurite is to project can include selecting the site based on a pairwise trunk angle distribution that is characteristic of neurons of a morphological type. Each of the successive plurality of distances can be between 0.5 and 3 micrometers apart.

In another aspect, methods, systems, and apparatus, including computer programs encoded on a computer storage medium, for generating model neurons are described. For example, such a method includes receiving a plurality of descriptions of branches of dendrites of one or more neurons and generating a collection of model neurites. Each of the descriptions characterizes, for an individual branch, i) a distance from a cell body at which the individual branch first bifurcates and ii) a distance from the cell body at which the individual branch actually terminates. Generating the collection of model neurites includes repeatedly selecting a description of a branch from the plurality and probabilistically generating a topology of a model neurite based on the selected description. The probabilistic generation of the model neurite includes deciding whether to bifurcate, terminate, or continue the model neurites at different positions based on the selected description.

This and other aspects can include one or more of the following features. Generating the collection of model neurites can include foreclosing selection of any description from the collection more than once. In response to a determination that a first of the model neurites is to bifurcate, a branch can be generated by selecting a description of a branch from the plurality and probabilistically generating the topology of the branch based on the selected description. Each of the descriptions can further characterize an angle between daughter branches at a bifurcation. The generation of model neurons can include calculating a direction in which daughters emerge from the bifurcation by assuming that each daughter branch emerges from a parent branch at a same angle. The generation of model neurons can include calculating a direction in which daughters emerge from the bifurcation by assuming that a first daughter branch continues in a same direction as a parent branch. Each of the plurality of descriptions of branches comprises a Topological Morphology Descriptor. The generation of model neurons can include assigning model neurites to cell bodies, wherein a number of the model neurites assigned to each of the cell bodies comprises a value that is characteristic of neurons of a morphological type. The generation of model neurons can include assigning sizes to the cell bodies, wherein the sizes are characteristic of the neurons of the morphological type. The generation of model neurons can include assigning diameters to terminations of the model neurites, wherein the diameters of the terminations are characteristic of terminations in the neurons of the morphological type. The generation of model neurons can include defining diameters of the model neurites, wherein the diameters are characteristic of the neurons of the morphological type. Generating the collection of model neurites can include the first aspect.

The details of one or more implementations are set forth in the accompanying drawings and the description below. Other features and advantages will be apparent from the description and drawings, and from the claims.

### DESCRIPTION OF DRAWINGS

FIG. 1 is a schematic representation of a process for generating models of neuronal dendrites.

FIG. 2 is a comparison of biological pyramidal cells and synthesized pyramidal cells of different rodent cortical morphological types from layers 2 to 6.

FIG. 3 is a comparison of biological interneurons and synthesized interneurons of different rodent cortical interneurons of layers 1 to 6.

FIG. 4 schematically illustrates how a rooted tree is transformed into a corresponding persistence barcode.

Like reference symbols in the various drawings indicate like elements.

### DETAILED DESCRIPTION

Neuronal morphologies determine the connectome and enable the dynamical properties of the brain. The computational generation of digital morphologies that reproduce the anatomical and electrical characteristics of biological neurons is therefore a vital step towards the reconstruction and simulation of physiologically realistic brain networks. However, the principles that determine how dendritic and axonal arbors take shape are still largely unknown.

A generative algorithm for neurons that accurately reproduces the diversity of biological morphologies for different cell types is therefore essential for the reconstruction and simulation of biophysically accurate computational models of the brain. The present synthesis algorithm can use the Topological Morphology Descriptor, TMD described by Kanari et al (Neuroinform. 16: 3-13 2018, https://doi.org/10.1007/s12021-017-9341-1, the contents of which are incorporated herein by reference) to demonstrate that by combining the topological and geometric properties of neurons, neurons that are indistinguishable from their biological counterparts, can be computationally generated, i.e., synthesized. The synthesis averts significant limitations of previous techniques, and is thus suitable for the efficient generation of large numbers of unique morphologies to populate biologically realistic brain networks.

A fundamental problem of neuronal synthesis is the difficulty to capture and recreate the correlations between morphological features from the few available reconstructions of a morphological type. These inter-dependencies of morphometrics arise from complicated developmental processes, which take place over many spatial and temporal scales. Previous synthesis models have addressed this problem in various ways.

Biophysically accurate models simulate detailed neural growth by taking into account the known molecular mechanisms that contribute to the development of neurons, capturing the correlations in the cellular growth. Although these models are very important for understanding the biological mechanisms that govern neuronal development, they focus on the microscopic scale of growth and have a large number of parameters. As a result, they are not sufficiently efficient for the computational synthesis of large numbers of neurons at whole brain length-scales.

Phenomenological models are based either on fundamental mathematical principles or on statistical sampling of the morphological distributions. Mathematical models with few parameters focus on a specific growth mechanism to study the effect of different factors on neuronal growth, such as spatial embedding, minimization of wiring cost, and self-referential forces. These models provide good intuition about the selected mechanisms involved in neuronal growth, at the price of not generalizing well to a large variety of cell types without appropriate adjustments to the algorithms.

On the other hand, statistical models that are based on sampling from a set of morphological properties can generate cells of a given morphological type with high accuracy but often disregard feature correlations as the sampling is performed independently. Even when correlations are explicitly identified, manual selection of feature dependencies is required, which renders statistical approaches computationally expensive and hard to generalize.

The synthesis described herein is based on the topological profile of a branching morphology, which implicitly reproduces key correlations between morphological features. The persistence barcode of a neuron, which encodes the start and end path distances of all branches within a tree, is used for the definition of the branching and termination probabilities. A set of additional morphological features (soma size, trunk directionality and thickness of branches) are used to reproduce properties that are orthogonal to the branching properties.

The topological neuron synthesis can synthesize dendrites of a large variety of inhibitory and excitatory cell types. Dendrites that are statistically indistinguishable from the biological reconstructions can be generated for a variety of morphological types. The computationally synthesized cells also reproduce the electrical behavior of the biological reconstructions. The synthesis can be generalized for the generation of other cell types by applying a mathematical transformation to reproduce varying cortical thickness.

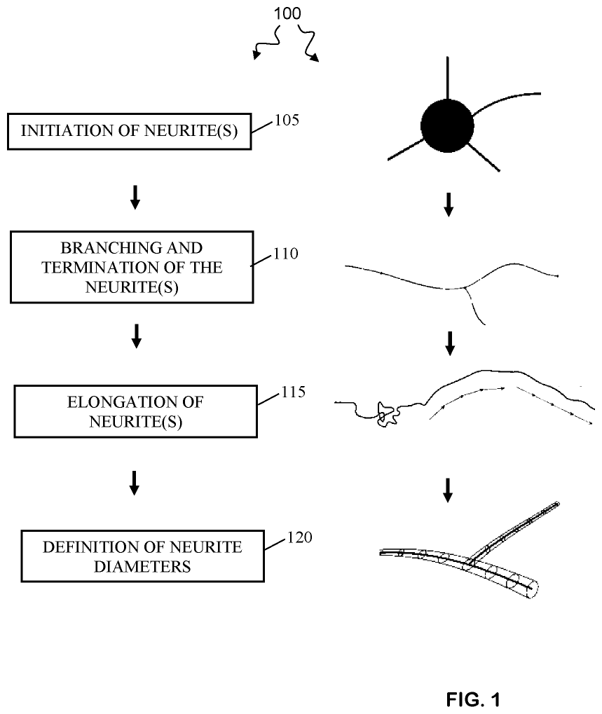

As represented schematically in FIG. 1, the synthesis process 100 can include:

a) initiation 105 of neurite(s);

b) branching and termination 110 of neurite(s);

c) elongation 115 of neurite(s); and

d) definition 120 of neurite diameter(s).

In an example initiation 105, the cell body (i.e., the soma) can be the first part of a neuron to be generated. In some implementations, the cell body is modeled as a sphere. The radius of the sphere can be sampled from a biological distribution of cell body sizes. In some implementations, the number of neurites per cell body can be sampled from the biological distribution of the corresponding cell type. Each neurite is initialized with a “trunk” (i.e., the initial branch of the tree) and a description of a branch of a dendrite of a biological neurons can be selected. As discussed below, the description can be a barcode from a Topological Morphology Descriptor.

The direction of the neurite protrusion from the soma can also be determined. For example, for some neurites, the initial direction is trivially determined from the biological neurons that are being synthesized. For example, cortical apical dendrites typically grow towards the pia. In contrast, the outgrowth directions of neuronal processes of basal dendrites are correlated. This correlation can be captured in the pairwise trunk-angle distribution that depends on the morphological type and that can be used for the initiation of neurites on the soma surface. For basal dendrites, the initial point of a single neurite can be randomly sampled on the soma surface, then the other dendrites can be added successively in places that respect the pairwise trunk-angle distribution.

In any case, each neurite trunk consists of a point on the soma surface and an initial direction that is normal to the soma surface. In some implementations, the positions of the trunks can define the soma shape in the model. For example, a pyramidal soma of excitatory cells can be defined based on an apical dendrite that points towards the pia. As another example, a spherical soma can be defined for interneurons based on the homogeneous positioning of trunks on the surface of their cell bodies.

In an example branching and termination 110, growth can take place stepwise in a loop. Each branch of the tree can be elongated as a directed random with memory. At each step a growing tip can be assigned probabilities to bifurcate, to terminate, or to continue. The probabilities can depend on the path distance from the soma. In implementations that use a barcode from a Topological Morphology Descriptor, the probabilities are defined by the bars of the topological profile of the associated morphological type. Once a bar is used, it is removed from the barcode. The branching and termination process terminates when all the bars of the input barcode have been used.

In some implementation of a branching and termination 110, a discrete random tree is generated. The tree only includes bifurcations, terminations and continuations. When the tree is grown stepwise, the possible values for the number of offspring at each are:
zero (i.e., termination),
one (i.e., continuation), or
two (i.e., bifurcation).
   At each step, a number of offspring is independently sampled from a probability distribution. In general, the bifurcation/termination probabilities will depend on the path distance of the growing tip from the soma. For example, in implementations that use a

Topological Morphology Descriptor, each growing tip is assigned a bar that is sampled from the barcode. The bar includes a starting radial distance B, an ending radial distance T, and a bifurcation angle A. At each step, uses the respective probabilities to determine whether to bifurcate and whether to terminate. If the growing tip does not bifurcate or terminate, then the branch continues to elongate. The bifurcation probability depends on the starting path distance B. In some implementations, as the growing tip gets closer to the radial distance B, the bifurcation probability increases exponentially until it reaches the highest possible value (1.0). Similarly, the termination probability depends exponentially on the ending radial distance T.

The termination and bifurcation probabilities are sampled from an exponential distribution exp(−λx), where x is distance from the soma and is a free parameter. A relatively large λ results in a steep exponential distribution. This results in cells that are very close to the biological neuron. The variance in the synthesized cells may be too small. In contrast, a relatively small λ yields in cells that are almost random, since of the bifurcation, termination, and continuation decisions will be almost independent of the characteristics of the biological neuron. Thus, in some implementations, the value of the parameter λ is between 10% and 1000% of a separation between successive distances at which the decisions whether to bifurcate, terminate, or continue are made, for example, between 30% and 500%. In some implementations, of the parameter λ is equal to the step size. With a parameter λ within these ranges, the bifurcation and termination points are stochastically chosen but are strongly correlated with the biological neurons.

In some implementations, including implementations that use a Topological Morphology Descriptor, the bifurcation and termination probabilities are correlated. For example, the correlation of these probabilities is captured in the structure of the barcode of a Topological Morphology Descriptor. When the growing tip bifurcates, the corresponding bar can be removed from the input Topological Morphology Descriptor to exclude re-sampling of the same conditional probability. This keeps a record of the neuronal growth history and reproduces the biological branching structure. In the event of a termination, the growing tip is deactivated, and the bar that corresponds to this termination point is similarly removed from the input TMD.

At a bifurcation, two new branches are generated. The directions of the daughter branches depend on the bifurcation angle A. Depending on the neurite type, different rules can be used to determine the bifurcation angles. For example, the bifurcation of basal dendrites can follow the biological bifurcation angle distribution. The apical tree is separated into two parts, namely:
the apical tuft, which is the densely branched subtree that is proximal to the cortical surface, and
the obliques, which are the shorter branches that emerge closer to the soma.
   The apical tuft is separated from the obliques by the “apical point.” The apical point is the distance that maximizes the separation between the two modes of the bar distribution. The apical point can be accurately identified, e.g., based on the persistence barcode of the apical tree. For the apical dendrites, different branching behaviors need to be adopted for the tuft and the obliques. Before the apical point, one of the branches, the major branch, follows the targeting direction (usually the orientation towards the pia). Once the apical point is reached, the apical tufts bifurcate according to the distribution of the biological bifurcation angles.

In an example elongation 115 of neurite(s), each synthesized neurite is grown segment by segment. A segment is a pair of consecutive points in the neuronal tree that determine a vector of length L and with direction D_{segment}. Direction D_{segment} can be specified, e.g., by a unit vector. In some implementations, the segment length L is constant. In some implementations, the segment length L is between, e.g., 0.3 and 5 microns, for example, between 0.5 and 3 microns, for example, equal to one micron. In some implementations, the direct of each segment is a weighted sum of three unit vector terms, namely:
the cumulative memory of the directions of previous segments within a branch M,
a target vector T, and
a random vector R.
   The memory term is a weighted sum of the previous directions of the branch with the weights decreasing with distance from the tip. As long as the memory function decreases faster than linearly with distance from the growing tip, a variety of different forms of the cumulative memory M can be used. The target vector T can be defined at the beginning of each branch and can depends on the biological branch angles. The random component R can be a vector of fixed length that is sampled uniformly from three-dimensional space at each step. In some implementations, the growth of each branch is independent of that of other branches. The tortuosity of the path is defined by three parameters, namely:

D_{segment}=ρR+τT+μM;

where ρ+τ+μ=1.
An increase of the randomness weight ρ results in a highly tortuous branch, approaching the limit of a random walk when ρ=1. If the targeting weight τ=1, the branch will be a straight line in the target direction. Different combinations of the three parameters (ρ, τ, μ) can generate more or less meandering branches and can reproduce the large diversity of dendritic sections.

In an example definition 120 of neurite diameter(s), the diameters of the tree are assigned based on diameter distributions sampled from the biological reconstructions—rather than actual measurements. Despite the great progress in imaging techniques that enables the generation of large numbers of reconstructions, their resolution is still too limited to allow for accurate determination of diameters. Thus, in some definitions 120 of neurite diameter(s), diameters are computationally inferred from sparse datasets of biological reconstructions.

In some implementations, the original diameters of the reconstructed cells are used as input for the synthesis algorithm. The reconstructions can be analyzed, e.g., using NeuroM, to extract the morphometrics related to the thickness of neurons.

Examples of these morphometrics include:
taper rate within a section, T_{R},
diameters of the termination, Dₜᵢₚ, and
trunk diameters, Dₜᵣᵤₙₖ of a tree.
   These values can be used to assign diameters independently to each synthesized dendrite.

In some implementations, the assignment process starts from the tips of the tree and assigns diameters to the termination points sampled from the biological distribution Dₜᵢₚ. Then, the tree is traversed from the tips towards the root and the diameters are increased according to the biologically sampled taper rate T_{R} so long as the new diameter remains less than a sampled maximum diameter, i.e., the trunk diameter Dₜᵣᵤₙₖ.

When the diameters of all the children of a section have been computed, the parent section can be assigned a diameter according to the Rall ratio which is chosen to be RR=3/2, namely:

dₚₐᵣₑₙₜ=(d₁^{RR}+d₂^{RR}+. . . )^{1/RR}

This approach yields a distribution of diameters that is statistically similar to the original distribution. In addition, the average diameter of the synthesized cells corresponds to the average biological diameters. The synthesized diameters monotonically decrease with distance from the soma, a property that ensures that basic physical principles of dendrites are reproduced.

The results of neuronal synthesis using synthesis process 100 can be seen in a comparison of biological and synthesized dendritic shapes. In particular, there are two major types of cortical cells, which are distinguished based on their functional roles: the excitatory cells and the inhibitory cells. Excitation is mainly mediated by the pyramidal cells, with the exception of the spiny stellate cells of layer 4, and use glutamate as a neurotransmitter. Inhibition is mediated by the interneurons, which use GABA as a neurotransmitter to regulate cortical activity. Synthesis process 100 can be used for the computational synthesis of dendrites of both interneurons and pyramidal cells of a large variety of morphological types. Because these two major cell classes present distinct morphological properties, the synthesized pyramidal cells and interneurons will be studied independently. Interneurons that have only basal dendrites, i.e. dendrites that emanate from the base of the cell body and are localized mainly around the soma, are less complex than pyramidal cells, which also have apical dendrites. The apical dendrites reach to higher cortical layers, typically ascending towards the pia, and present a wider diversity of shapes.

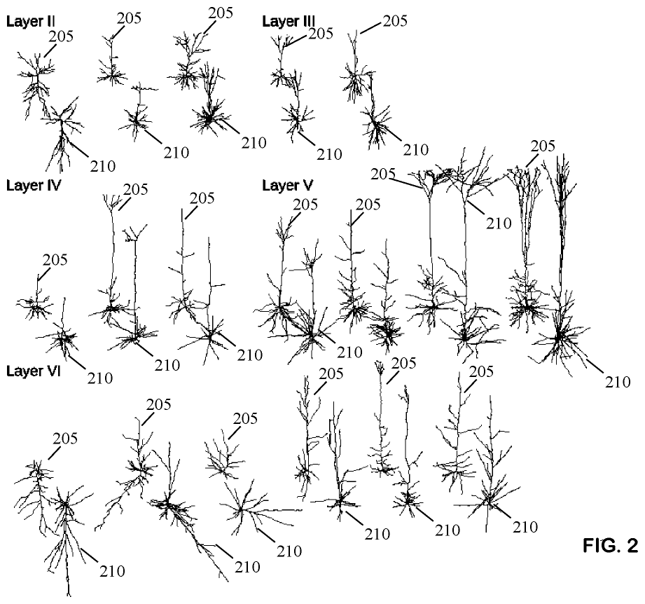

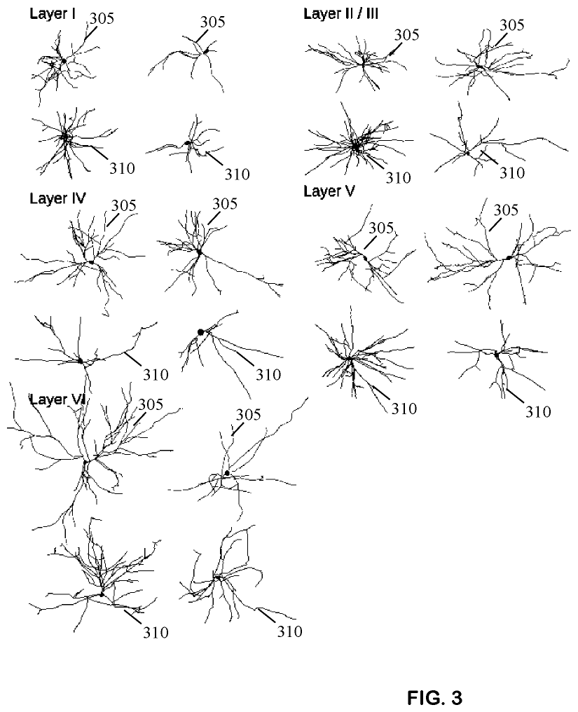

FIG. 2 is a comparison of biological pyramidal cells 205 and synthesized pyramidal cells 210 of different rodent cortical morphological types from layers 2 to 6. FIG. 3 is a comparison of biological interneurons 305 and synthesized interneurons 310 of different rodent cortical interneurons of layers 1 to 6. As shown, biological relevant models can be obtained.

As discussed above, implementations of synthesis process 100 can use Topological Morphology Descriptors. Topological Morphology Descriptors include a persistence barcode Barcode from a tree T. The generation of Topological Morphology Descriptors encodes the branching pattern of the morphology into a topological representation. The local fluctuations with little information content, such as the position of the nodes between branch points, are discarded. Thus the computational complexity of the tree is significantly reduced. The TMD algorithm couples the topology of the branching structure with the geometric properties of the tree (for example, the radial distance from the soma). The over-all shape of the tree is thus encoded into a single descriptor.

The process for generating Topological Morphology Descriptors receives a set of branch points (nodes with more than one child) and leaves (nodes with no children) of the tree as inputs, and produces a multi-set of intervals, i.e., bars, on the real line known as a persistence barcode.

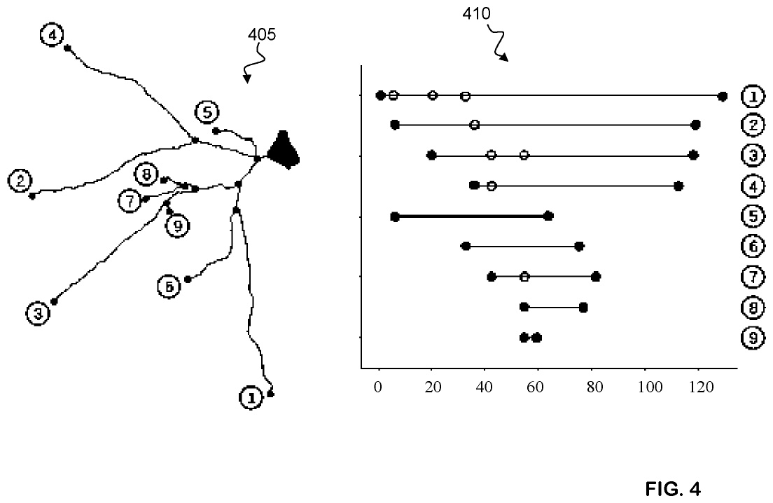

FIG. 4 schematically illustrates how a rooted tree 405 is transformed into a corresponding persistence barcode 410. The correspondence between the tree 405 and its extracted barcode 410 is highlighted by denoting the same branches in both using circled numbers (i.e., 1, 2, 3, . . . in circles). As shown, each bar in barcode 410 represents a branch in tree 405, including the position of bifurcations and terminations. Each bar thus encodes the lifetime of a component in the underlying structure, identifying when a branch is first detected (birth) and when it connects to a larger subtree (death). The representation in persistence barcode 410 thus greatly simplifies the mathematical complexity of rooted tree 405.

To generate a barcode 410 from a rooted tree 405, the rooted tree 405 is input with a functionf defined on the set of the tree's nodes. In some implementations, the functionfis the radial distance from the soma. Each branch within rooted tree 405 is transformed into a bar in the persistence barcode 410 encoding its start and end radial distance. Starting from the tips, the radial distance of two siblings, i.e., branches starting at the same bifurcation point, are compared. For example, branches 1 and 6 are siblings. Branch 1 has a larger radial distance from the soma that branch 6. Hence, branch 1 persists though the junction between branches 1 and 6, whereas the smaller sibling branch 6 “dies” at that same junction.

In some implementations in which Topological Morphology Descriptors are used in a systhesis process 100, persistence barcodes are generated from a set of neuronal reconstructions. In some implementations, along with the topological profiles of the neurons, a set of basic morphometrics can also be extracted. For example, morphometrics related to the features of the soma and the thickness of the tree can be extracted. For example, soma parameters such as, e.g.,
soma diameters SS,
the number of neurites NN of a specific type within a neuron, and
pairwise angles PA between neurites can be extracted.

As another example, diameter parameters such as, e.g.,
diameters of the tips (or terminations) TD of the neurite,
taper rates TR that define the tapering, i.e., the difference of the diameters normalized by the length, within a section of the neurite (D_{final}−Dᵢₙᵢₜᵢₐₗ)/length. This value can be adjusted so that the average value of the diameters within a section is preserved,
Rall ratios n that define the ratio between the diameter of a parent and its children. The Rall ratio n is the exponent for which Dⁿ=d₁ⁿ+d₂ⁿ+ . . . at a branch point, where dᵢ are the diameters of the children and D is the parent diameter, and
maximum diameters MD of each neurite-type can be extracted.

In addition to or instead of biological inputs, a set of user-defined parameters can also be used. For example,
the weight of the targeting bias ti that is used for the elongation of a section,
the weight of the random component p that is used for the elongation of a section,
the definition of a center of the soma c and the starting point for the growth of the neuron, and
the radial distance of the apical point from the soma D_{apical}, which is used to modify the oblique branching method used during the growth of an apical tree to the tuft branching method,
   can be defined by a user.

In synthesis process 100, a “soma” is the cell body and described as a sphere S_{ds}^{c} of diameter dₛ and center c. A “neurite” is a neuronal tree. A “neurite point” is given by a set of parameters (x; y; z; d), where x, y, z are the coordinates in 3D space and d is the diameter that represents the thickness of the neurite at that point. A “neurite section” is a list of points in the neurite that are between two branch points or between a branch point and a termination. A “section” can also be referred to as a “branch.” “Neurite tips” are the collection of termination points of a neurite. A “neurite trunk” is the initial section of a neurite, as it emerges from the soma. The vector “Vect” is a spherical unit vector, which is equivalently represented by a pair of angles and defines a direction, or orientation, in 3D space.

As described above, the persistence barcodes that are extracted from a neuronal tree are used as input for the synthesis process 100. In some implementations, the persistence barcodes can be enhanced with the bifurcation angles of their corresponding components. At the point where the component terminates, the bifurcation angle A is encoded with its parent. Therefore, for each component of the tree T, a Bar =(B; T; A) is defined, where B is the initial radial distance of the component, T is the terminal radial distance and A is the bifurcation angle at which the component emerges from its parent. In order to use a population of neurons as input for synthesis, the barcodes of all the biological trees are extracted.

BARcodes={Barcodeⱼ|1≤j≤n},

where n=the number of neuronal trees in the biological population.

This distribution of persistence barcodes is sampled during the growth of a neuron and a single barcode, which corresponds to a biological tree is used for the generation of a synthesized neuronal tree.

Barcodeⱼ={Barᵢ=(Bᵢ; Tᵢ; Aᵢ)|1≤i≤bⱼ},

where bⱼ=number of components (bars) of Barcodeⱼ.

As discussed above, in some implementations, a number of independent morphometrics can be measured—in addition to the persistence barcodes which encode the topology of the neurites. For example, morphometrics that define the size of the cell body, or soma can be measured. The soma can initially be modeled as a sphere and therefore a center and a diameter are sufficient to describe it. In some implementations, the center can be defined by a user, whereas the diameter is sampled from the corresponding biological distribution SS.

Also as discussed above, in some implementations, parameters that are not measured as input from the biological dataset can be input by a user. Examples include ti and p, which define the properties of the elongation within a branch. The center of the soma c can also be a user-input parameter to allow the user to control the initial position of the cell. The apical point distance D_{apical} can also be a user-defined parameter.

In a synthesis process 100 that uses s Topological Morphology Descriptors, the generation of a soma and initiation 105 of neurite(s) can first determine an initial direction for neurite growth. For some neurites, the initial direction is well defined; for example cortical apical dendrites typically grow towards the pia. In basal dendrites, the outgrowth direction of pairs of basal dendrites are correlated. When such pairwise angles distribution is used when initiating neurites, the synthesized dendrites respect the biologically acceptable distances.

In a synthesis process 100 that uses Topological Morphology Descriptors, the elongation 115 of neurite(s) can proceed segment-by-segment, where each segment has a length L and a direction D_{segment}. The direction of a new segment is defined by a unit vector as a weighted sum of three components, namely: the cumulative memory M of the directions of the previous segments within a branch, the target vector T, which is chosen at the beginning of a branch, and the randomly sampled vector from the unit sphere R. The direction of the new segment is:

D_{segment}=ρ*R+τ*T+μ*M.

In some implementations, the segment length is constant and equal to one micron L=1 μm, the weights that define the components of the next point are also normalized to one ρ+τ+μ=1. As a result, only two of the input parameters need to be defined.

As the randomness weight ρ is increased the branch becomes more tortuous, approaching the limit of a simple random walk when ρ=1. On the other hand, an increase of the targeting weight τ results drive branches towards a straight line the target direction. The memory weight μ has a more complicated effect on the generated branch. For high values of the targeting weight τ the line is already straight so the effect of larger memory weights μ is not significant. For lower values of the targeting weight τ the memory generates shapes of larger curvature but lower local randomness since correlations between segment directions are preserved for longer distances.

In a synthesis process 100 that uses Topological Morphology Descriptors, the branching and termination 110 of neurite(s) can include assigning each growing tip a barcode that includes a starting radial distance B, an ending radial distance T, and a bifurcation angle A. It is determined whether the active tip bifurcates based on the bifurcation probability. If a bifurcation does not occur, it is determined whether the active tip terminates based on termination probability. If the active tip does not bifurcate or terminate, then the branch continues to elongate. The bifurcation probability depends on the starting radial distance B and the termination probability depends exponentially on the ending radial distance T.

As the active tip approaches the radial distance B, the bifurcation probability increases according to an exponential distribution exp (−λx) until it reaches the highest possible value 1.0, i.e., when the tip exceeds the target radial distance. The rate parameter λ of the exponential distribution exp (−λx) controls the bifurcation probability and the termination probability. The rate parameter λ is thus important for the generation of biologically relevant neurites. A relatively steep exponential distribution (high value of λ) generates cells that resemble the biological input and thus the variability of the synthesized cells is small. On the other hand, a relatively low value of λ generates cells that are almost random, since the dependence on the input persistence barcodes is minimal. If the value of parameter λ is of the order of the step size, the bifurcation will occur within a few steps from the target radial distance. As a result, the generated shapes will differ from the input branching structure, introducing the necessary variability, but will preserve the overall shape of the input tree and generate biologically acceptable structures.

In the event of a bifurcation, two new branches are generated. The directions of the daughter branches depend on the input bifurcation angle A. Different branching methods can be used, including a symmetric branching, a biased branching, and a composite branching. In symmetric branching, the two daughter branches emerge at the same angles from their parent branch. The bifurcation angle is split in two and equally distributed among the daughter branches. In biased branching, one of the daughter branches continues to grow towards the direction of its parent and therefore the split is asymmetric. Composite branching is a combination of the symmetric and the biased methods. In composite branching, two biological angles are used, namely, the bifurcation angle A which defines the angle between the daughter branches, and the parent-daughter angle which defines the angle between the parent and one of the daughter branches.

The following tables are pseudocode representations of an example synthesis process 100 that uses Topological Morphology Descriptors. In particular, Table 1 gives an overview of an example of such a synthesis process 100.

TABLE 1
Input:

| | |
|---|---|
| Bio = {SS, NN, PA, TD, TR, RR, MD} | [symbol] (see Biological distributions) |
| Param = {c, τ, ρ} | [symbol] (see Input Parameters) |
| BARcodes | [symbol] (see Biological barcodes) |

function SAMPLE(distr) := draws from input distribution

| | |
|---|---|
| Generate a Soma and Neurites using (Alg 2, Bio, c) | [symbol] |

```
  (each neurite is initialized with a point on the soma surface, which defines
  a direction dir₁)
  for neurite in Neurites do
    Active ← neurite's initial point
    Barcode = SAMPLE(BARcodes)
    Sort Bars in Barcode from longest to shortest
    Initialize first section S₁ with the longest Bar₁
    while Active sections do
```

| | |
|---|---|
| for Section Sₖ in Active do | [symbol] (a section gets a target direction |

```
  dirₖ and a bar Barₖ)
        Grow a section using (Alg 3, dirₖ, Barₖ = (Bₖ, Tₖ, Aₖ))
```

| | |
|---|---|
| Remove Barₖ from Barcode | [symbol] (each Bar can be used only |

```
  once)
        if status = Bifurcate then
          Generate children using (Alg 4, Barcode, Barₖ, dirₖ)
          Add children to Active sections
        else if status = Terminate then
          Section growth terminates
        Remove current section Sₖ from Active
  Generate accurate diameters using (Alg 5, Bio)
Output:
  A neuron: a set of points and their connectivity.
```

Table 2 is an example generation of a soma and initiation 105 of neurite(s) in a synthesis process 100.

TABLE 2
Input:

| | |
|---|---|
| Bio = {SS, NN, PA, c} | [symbol] (see Biological distributions) |

```
  function SAMPLE(distr) := draws from input distribution
  dₛ = SAMPLE(SS)
  Soma is a sphere of diameter dₛ and center c: S_{d}_{ₛ}^{c}
  #neurites = SAMPLE(NN)
  Create first neurite N₁ on S_{d}_{ₛ}^{c} surface at random direction, Vect₁
  The first point of the neurite P₁¹ is on S_{d}_{ₛ}^{c} surface
  for Neurite (Nᵢ|2 ≤ i ≤ #neurites) do
    Vectᵢ = Vectᵢ₋₁ + SAMPLE(PA)
    First point of Nᵢ is Pᵢ¹ which corresponds to Vectᵢ
Output:
  A soma S_{d}_{ₛ}^{c} and the initial points of each neurite Nᵢ
```

Table 3 is an example elongation elongation of neurites 110 in a synthesis process 100.

TABLE 3
Input:
τ, ρ, dir, Barₖ = (Bₖ, Tₖ, Aₖ), x₀

| | |
|---|---|
| μ = 1 − τ − ρ | [symbol] (Normalization of weights to 1) |

```
  function RD(point) := radial distance of point from soma
  n = 1
  status = Continue
  while status is Continue do
    random = random direction sampled uniformly in a unit sphere
    memory = direction from the weighted sum of previous directions
    x_{i≤n}
    xₙ₊₁ = xₙ + ρ * random + τ * dir + μ * memory
    if Check Pr(Bifurcate | RD(xₙ₊₁), Bₖ) then
      status = Bifurcate
    else if Check Pr(Terminate | RD(xₙ₊₁), Tₖ) then
      status = Terminate
    else
      status = Continue
Output:
    A section and a status which is either a bifurcation or a termination.
```

Table 4 is an example of bifurcation of a neurite in a synthesis process 100.

```
TABLE 4
Input:
  Barcode, Barₖ = (Bₖ, Tₖ, Aₖ)
  function SPLIT(vect) := returns two new unit vectors dir₁, dir₂ from the
  input unit vector
  dir₁, dir₂ = SPLIT(Aₖ)
  Find next available index i in Barcode for which min(Bᵢ)
  Generate child section 1: ← dir₁, Bar₁ = (Bᵢ, Tₖ, Aᵢ)
  Generate child section 2: ← dir₂, Bar₂ = (Bᵢ₊₁, Tᵢ₊₁, Aᵢ₊₁)
Output:
  Two new sections, each initialized with a direction dir and a Bar.
```

Table 5 is an example of definition of neurite diameters 120 in a synthesis process 100.

TABLE 5

| Input: | |
|---|---|
| Bio : TD, TR, RR, MD (see Biological distributions) | |
| function SAMPLE(distr) := draws number from distribution | |
| for all Neurite tips do dₜᵢₚ = SAMPLE(TD) | |
| Active ← tips | |
| while Active do | |
| for Section in Active do | |
| taper = SAMPLE(TR) | |
| for Point in Section do | [symbol] From termination to the root |

```
        d_{new} = dₙ₊₁ + taper * length
        if d_{new} ≤ MD then
          dₙ = d_{new}
        else
          dₙ = dₙ₊₁
      Remove Section from Active
      if all siblings 1, 2, . . . computed then
        n = SAMPLE(RR)
        Dₚₐᵣₑₙₜ = (d₁ⁿ + d₂ⁿ + . . . )^{(1/n)}
        Add parent Section to Active
Output:
  Assigns new values to the diameters of the neuron.
```

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. A computer-implemented method for synthesizing models of branching morphologies, the method comprising:
receiving a persistence barcode that characterizes a plurality of biological branches, wherein bars in the persistence barcode represent positions of bifurcations and terminations of the biological branches, wherein each bar in the persistence barcode characterizes a single of the biological branches; and
generating a collection of model branches, including repeatedly selecting a bar from the persistence barcode and probabilistically generating a topology of a model branch based on the selected bar,
wherein the probabilistic generation of the model branch includes deciding whether to bifurcate, terminate, or continue the model branch at different positions, wherein the bifurcation probability is based on a position of a bifurcation in the selected bar and the termination probability is based on a position of a termination in the selected bar.

2. The method of claim 1, wherein the persistence barcode characterizes the biological branches of a single tree.

3. The method of claim 2, wherein the bars in the barcode encode radial distance from a source of a trunk of the tree.

4. The method of claim 1, wherein, in response to deciding to bifurcate a first branch based on a first selected bar, the method includes selecting a second bar from a subset of the bars in the persistence barcode, wherein the subset of the bars excludes the first selected bar.

5. The method of claim 1, wherein:
at least some of the bars of the persistence barcode include a bifurcation angle that characterizes an angle at which a component emerges from the biological branch characterized by those bars; and
the method further comprises determining directions of daughter branches based on the bifurcation angles.

6. The method of claim 1, wherein the bifurcation probability and the termination probability are sampled from an exponential distribution exp (−λx), wherein:
x is the position of the bifurcation or the position of the termination and
λ is between 10% and 1000% of a separation between successive distances at which the decisions whether to bifurcate, terminate, or continue are made.

7. The method of claim 1, wherein the biological branches are neurites.

8. A computer-implemented method for generating model neurons, the method comprising:
selecting, on a cell body of the model neuron, a site from which a model neurite is to project from the cell body; and
probabilistically generating the model neurite, including, for each of a successive plurality of distances from the cell body, deciding whether to
i) bifurcate the neurite,
ii) terminate the neurite, or
iii) continue the neurite for another step,
wherein a probability of bifurcation, a probability of termination, and a probability of continuation are functions of the distance from the cell body.

9. The method of claim 8, wherein the probability of bifurcation is calculated based on a distance from the cell body at which a first branch of a neuronal dendrite first bifurcates.

10. The method of claim 8, wherein the probability of termination is calculated based on a distance from the cell body at which the branch of the neuronal dendrite actually terminates.

11. The method of claim 8, wherein the probability of bifurcation and the probability of termination are sampled from an exponential distribution exp (-Xx), wherein:
x is the distance from the cell body and
λ is between 10% and 1000% of a separation between successive distances at which the decisions whether to bifurcate, terminate, or continue are made.

12. The method of claim 8, wherein selecting the site from which the neurite is to project comprises selecting the site based on a second site from which a second neurite projects from the cell body.

13. The method of claim 12, wherein selecting the site from which the neurite is to project comprises selecting the site based on a pairwise trunk angle distribution that is characteristic of neurons of a morphological type.

14. The method of claim 8, wherein each of the successive plurality of distances is between 0.5 and 3 micrometers apart.

15. A computer-implemented method for generating model neurons, the method comprising:
receiving a plurality of descriptions of branches of dendrites of one or more neurons, wherein each of the descriptions characterizes, for an individual branch,
i) a distance from a cell body at which the individual branch first bifurcates and
ii) a distance from the cell body at which the individual branch actually terminates; and
generating a collection of model neurites, including repeatedly selecting a description of a branch from the plurality and probabilistically generating a topology of a model neurite based on the selected description,
wherein the probabilistic generation of the model neurite includes deciding whether to bifurcate, terminate, or continue the model neurites at different positions based on the selected description.

16. The method of claim 15, wherein generating the collection of model neurites including foreclosing selection of any description from the collection more than once.

17. The method of claim 15, wherein, in response to a determination that a first of the model neurites is to bifurcate, a branch is generated by selecting a description of a branch from the plurality and probabilistically generating the topology of the branch based on the selected description.

18. The method of claim 15, wherein each of the descriptions further characterizes iii) an angle between daughter branches at a bifurcation.

19. The method of claim 18, further comprising calculating a direction in which daughters emerge from the bifurcation by assuming that each daughter branch emerges from a parent branch at a same angle.

20. The method of claim 18, further comprising calculating a direction in which daughters emerge from the bifurcation by assuming that a first daughter branch continues in a same direction as a parent branch.

21. The method of claim 15, wherein each of the plurality of descriptions of branches comprises a Topological Morphology Descriptor.

22. The method of claim 15, further comprising one or more of:
a) assigning model neurites to cell bodies, wherein a number of the model neurites assigned to each of the cell bodies comprises a value that is characteristic of neurons of a morphological type; or
b) assigning sizes to the cell bodies, wherein the sizes are characteristic of the neurons of the morphological type; or
c) assigning diameters to terminations of the model neurites, wherein the diameters of the terminations are characteristic of terminations in the neurons of the morphological type; or
d) defining diameters of the model neurites, wherein the diameters are characteristic of the neurons of the morphological type.

23. (canceled)

24. (canceled)
